(19) United States
(12) Patent Application Publication
(10) Pub. No.: US 2022/0227804 A1
(43) Pub. Date: Jul. 21, 2022

(54) COMBINATION OF A POLYHYDROXYLATED BILE ACID AND A FARNESOID X RECEPTOR AGONIST

(71) Applicant: QING BILE THERAPEUTICS INC., Vancouver (CA)

(72) Inventors: Victor LING, Vancouver (CA); Lin LIU, Vancouver (CA); Jonathan Ahab SHEPS, Vancouver (CA); Renxue WANG, Vancouver (CA)

(21) Appl. No.: 17/614,679

(22) PCT Filed: May 29, 2020
(86) PCT No.: PCT/IB2020/055097

§ 371 Date: Nov. 29, 2021

Related U.S. Application Data
(60) Provisional application No. 62/853,908, filed on May 29, 2019.

(51) Int. Cl. C07J 41/00; C07J 9/00; A61P 1/16

(52) U.S. Cl. CPC C07J 41/0061; A61P 1/16; C07J 9/005

(57) ABSTRACT

The present invention relates to, in part, a combination therapy of a polyhydroxylated bile acid and a farnesoid X receptor agonist. The present invention also provides, in part, a pharmaceutical composition comprising a farnesoid X receptor agonist and a polyhydroxylated bile acid in the preparation of a medicament for treating a biliary disorder or a gastrointestinal disorder.

### FIELD OF THE INVENTION

The present invention provides a combination therapy. More specifically, the present invention provides a combination therapy including a polyhydroxylated bile acid and a farnesoid X receptor agonist.

### BACKGROUND OF THE INVENTION

Bile is a complex secretion produced by the liver. It is stored in the gall bladder and periodically released into the small intestine to aid in digestion. Bile components include cholesterol, phospholipids, bile pigments, and various toxins that the liver eliminates through biliary/fecal exclusion. Bile salts are synthesized and actively secreted across canalicular membranes providing the osmotic force to drive the flow of bile. This is the rate-limiting step for bile formation. Bile flow is essential for liver detoxification, digestion, cholesterol metabolism, and absorption of lipid-soluble nutrients and vitamins.

Bile acids are critical as carriers for elimination of cholesterol from the body through biliary secretion and as a detergent for the ingestion of fatty acids and fat-soluble vitamins (1). Bile acids also play important roles in regulating cell apoptosis/survival (2; 3; 4; 5; 6) and in regulating gene expression through the farnesoid X receptor (FXR) (7; 8; 9; 10; 11; 12) in hepatocytes. Bile acids are synthesized in hepatocytes from cholesterol, secreted into the bile after being conjugated at the C24 position with glycine or taurine, reabsorbed in the small intestine, and recirculated back to hepatocytes through the portal vein. Canalicular secretion of bile acids from liver into the bile is a key process in the enterohepatic circulation of bile acids and its malfunction results in different hepatic diseases (1). If this process is disrupted, accumulation of bile acids often causes liver damage due to detergent effects. In humans, the bile acid pool circulates 6-10 times every 24 h, resulting in daily bile salt secretion of 20-40 g in about 400 ml (13; 14).

Common bile acids found in the bile of selected mammals include the following:

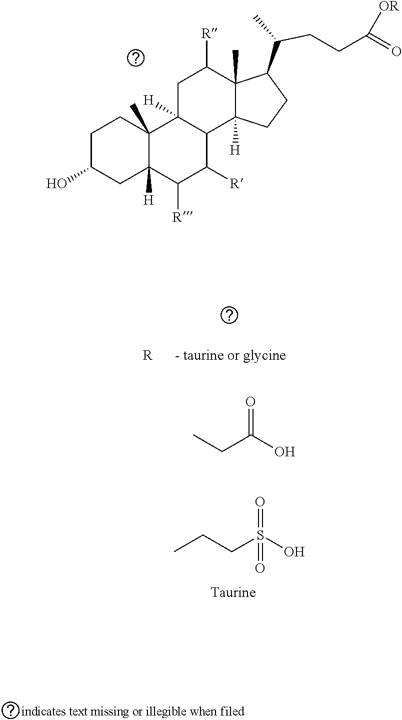

| Common Name | R [symbol] | R [symbol] | R [symbol] | Commonly found in species |
|---|---|---|---|---|
| Cholic acid | α-OH | α-OH | H | bear,cat,hamster,human |
| (3α7α12α) | | | | mouse,pig,rabbit,rat |
| [symbol] acid | α-OH | H | H | bear,hamster,human,pig |
| (3α7α) | | | | |
| Deoxcholic acid | M | α-OH | H | cat,human,rabbit |
| (3α12α) | | | | |
| Linsoodoxycholic acide | β-OH | H | H | bear |
| (3α7β) | | | | |
| Lithocholic acide | H | H | H | human,rat,mouse |
| (3α) | | | | |
| β- [symbol] acid | β-OH | H | β-OH | mouse,rat |
| (3α6α7β) | | | | |
| α- [symbol] acid | α-OH | H | β-OH | pig,mouse,rat |
| (3α6α7β) | | | | |
| Ω- [symbol] Muricholic acid | β-Oh | H | α-OH | mouse, rat |
| (3α6α7α) | | | | |

[symbol]

indicates data missing or illegible when filed

Some cholestatic conditions, such as Primary Biliary Cirrhosis, are treated by supplementation with a low-toxicity bile acid not normally found in human bile, ursodeoxycholic acid (UDCA).

Tetrahydroxylated bile acids (THBAs) are a class of bile acids that can stimulate bile flow while reducing the hydrophobicity of the bile acid pool, to treat biliary disorders and cholestatic conditions, for example, as disclosed in WO 2011/022838, published Mar. 3, 2011.

The farnesoid X receptor (FXR), also known as the bile acid receptor (BAR) or NR1H4 (nuclear receptor subfamily 1, group H, member 4) is a member of the nuclear receptor (NR) superfamily that is encoded by the NR1H4 gene in humans. FXR has been implicated in the regulation of bile acid synthesis, conjugation and transport (36)

Obeticholic acid (OCA), the 6α-ethyl derivative of chenodeoxycholic acid (CDCA), is an agonist of FXR. OCA efficiently inhibits bile acid synthesis, reducing circulating bile acids through FXR mediated gene regulation. OCA, as the drug product OCALIVA® is approved by the FDA for the treatment of primary biliary cholangitis (PBC) in adult patients. OCA has also been indicated for the treatment of PBC in combination with UDCA in adults with an inadequate response to UDCA, or as monotherapy in adults unable to tolerate UDCA. High-dose OCA in combination with UDCA has been reported to cause jaundice in those patients who do not benefit from UDCA monotherapy (15).

Tropifexor (TXR), 2-[(1R,5S)-3-[[5-Cyclopropyl-3-[2-(trifluoromethoxy)phenyl]-1,2-oxazol-4-yl]methoxy]-8-azabicyclo[3.2.1]octan-8-yl]-4-fluoro-1,3-benzothiazole-6-carboxylic acid, is an agonist of FXR that is under investigation for the treatment of cholestatic liver disease and nonalcoholic steatohepatitis.

### SUMMARY OF THE INVENTION

In one aspect, the present invention provides a pharmaceutical composition comprising i) a polyhydroxylated bile acid; and ii) an inoperable amount of a farnesoid X receptor agonist, in combination with a pharmaceutically acceptable carrier.

In some embodiments, the inoperable amount of a farnesoid X receptor agonist may be a subtherapeutic amount or a toxic amount. In some embodiments the ratio of the farnesoid X receptor agonist to the polyhydroxylated bile acid may be equal to or less than 1:100. In various embodiments, the farnesoid X receptor agonist may be obeticholic acid or tropifexor; the polyhydroxylated bile acid may be a tetrahydroxylated bile acid, such as 3α, 6α, 7α, 12α-tetrahydroxy-5β-cholan-24-oic acid; and/or the tetrahydroxylated bile acid may be a conjugated compound, such as a taurine or a glycine conjugate.

In an alternative aspect, the present invention provides a method of treating a biliary disorder or a gastrointestinal disorder by administering a composition as described herein to a subject in need thereof. In various embodiments, the biliary disorder may arise from cholestasis; the gastrointestinal disorder may be an inflammatory disorder.

In an alternative aspect, the present invention provides a method for reducing the toxicity of a farnesoid X receptor agonist by comprising i) a polyhydroxylated bile acid; and ii) a toxic amount of a farnesoid X receptor agonist, in combination with a pharmaceutically acceptable carrier.

In an alternative aspect, the present invention provides a method for enhancing the therapeutic effect of a farnesoid X receptor agonist by comprising i) a polyhydroxylated bile acid; and ii) an inoperable amount of a farnesoid X receptor agonist, in combination with a pharmaceutically acceptable carrier.

In various embodiments, the farnesoid X receptor agonist may be obeticholic acid or tropifexor; the polyhydroxylated bile acid may be a tetrahydroxylated bile acid such as 3α, 6α, 7α, 12α-tetrahydroxy-5β-cholan-24-oic acid; and/or the tetrahydroxylated bile acid may be a conjugated compound, such as a taurine or a glycine conjugate and/or the subject may be a human.

In an alternative aspect, the present invention provides the use of a composition as described herein in the preparation of a medicament.

In various embodiments, the composition may be used in treating a biliary disorder or a gastrointestinal disorder.

In an alternative aspect, the present invention provides an article of manufacture or a kit including a composition as described herein, together with instructions treating a biliary disorder or a gastrointestinal disorder.

This summary of the invention does not necessarily describe all features of the invention.

### BRIEF DESCRIPTION OF THE DRAWINGS

FIG. 1 shows bilirubin, ALT, ALP and total bile acids in the plasma of Mdr2^{-/-} mice under monotreatment conditions fed either 1% THBA (w/w), 0.03% OCA (w/w), or control diet for 17 weeks from 3 weeks of age to 20 weeks. Statistical significance by one-way ANOVA (Tukey's multiple comparison): *, P<0.05; **, P<0.01; ***, P<0.001; ****, P<0.0001.

FIG. 2 shows bilirubin, ALT, ALP and total bile acids in the plasma of Mdr2^{-/-} mice under treatments of different dose combinations of 1% THBA (w/w) with or without different doses of OCA (0.03%, 0.01% and 0.003% (w/w)), or control diet, for 4 weeks from 8 weeks to 12 weeks of age. Statistical significance by one-way ANOVA (Tukey's multiple comparison): *, P<0.05; **, P<0.01; ***, P<0.001; ****, P<0.0001.

FIG. 3 shows ALP in the plasma of Mdr2^{-/-} mice under treatments of different dose combinations of TXR (0.00001% and 0.000003% (w/w)), 1% THBA (w/w) with or without different doses of TXR (0.00001% and 0.000003% (w/w)), or control diet. Asterisks indicate statistical significance by one-way ANOVA (Tukey's multiple comparison) of each pair of TXR treatments against either THBA-fed or control diet groups. *, P<0.05; **, P<0.01, ***, P<0.001, and ****, P<0.0001.

### DETAILED DESCRIPTION

The present disclosure provides, in part, a combination therapy including a polyhydroxylated bile acid and a farnesoid X-activated receptor (FXR) agonist (the “claimed composition” or the “claimed combination”).

In some embodiments, the present disclosure provides a combination therapy including a tetrahydroxylated bile acid (THBA) and a farnesoid X-activated receptor (FXR) agonist.

In some embodiments, the polyhydroxylated bile acids can be agents of bile salt therapy to promote or improve biliary secretion, in combination with an FXR agonist, in subjects with biliary disorders or gastrointestinal (GI) disorders. A combination therapy according to the present disclosure can further be used in combination with additional known compounds, such as ursodeoxycholate or a variant or derivative thereof, to improve liver function and/or ameliorate a bile or GI disorder. In some embodiments, the claimed composition can result in reduced liver injury, for example in cholestatic diseases, or reduced intestinal injury, for example in inflammatory GI disorders.

In some embodiments, the claimed composition can reduce the toxicity and/or hydrophobicity of the bile acid pool. In some embodiments, the claimed composition can reduce the production of toxic bile acids, such as chenodeoxycholic acid (CDCA). In some embodiments, the claimed composition can stimulate bile flow.

In some embodiments, the claimed composition can reduce the toxicity of an FXR agonist. Accordingly, in some embodiments, the present disclosure provides a method for reducing the toxicity of a farnesoid X receptor agonist by administering a polyhydroxylated bile acid in combination with a toxic amount of a farnesoid X receptor agonist to a subject in need thereof, where the amount of the polyhydroxylated bile acid is sufficient to substantially counteract the toxicity of the farnesoid X receptor agonist. It is to be understood that some level of toxicity may be acceptable, as described herein. The reduction in toxicity can be compared, for example, in comparison to the toxicity of the farnesoid X receptor agonist when administered as a monotherapy. Toxicity can be measured using standard techniques as described herein or known in the art.

In some embodiments, the claimed composition can reduce the production of hydrophobic primary bile acids cholic acid (CA) and CDCA. In some embodiments, the claimed composition reduces the production of hydrophobic secondary bile acids lithocholic acid (LCA) and deoxycholic acid (DCA). The polyhydroxylated bile acids can be choleretic (possess bile flow-stimulating properties) when administered to a subject. In alternative embodiments, the polyhydroxylated bile acids can stimulate bile flow in any subject, for example, a subject not diagnosed with a GI or biliary disorder. By “stimulating bile flow” is meant increasing bile flow in a subject relative to a standard (e.g., standard levels of bile acid in an organism), or relative to the level of bile measured in the subject prior to administration of a combination therapy according to the invention. The increase may be a change of any integer value between 5% and 95%, or between 10% and 90%, or between 30% and 60%, or may be over 100%. As used herein, a subject may be a human, non-human primate, rat, mouse, cow, horse, pig, sheep, goat, dog, cat, etc. The subject may be a clinical patient, a clinical trial volunteer, an experimental animal, etc. The subject may be suspected of having or be at risk for having a GI or biliary disorder, be diagnosed with a GI or biliary disorder, or be a subject confirmed to not have a GI or biliary disorder. Diagnostic methods for GI or biliary disorders and methods for measurement of bile flow, as well as the clinical delineation of GI or biliary disorder diagnoses, are known to those of ordinary skill in the art.

Biliary Disorders

Biliary disorders include any disorder or condition that can be ameliorated, treated or prevented by the administration of a polyhydroxylated bile acid. Exemplary biliary disorders may include without limitation bile deficiency, bile toxicity, digestive disorders, impaired liver function, cholestasis, portal hypertension, etc. In some embodiments, biliary disorders include any condition that is known to be, or is expected to be, responsive to a therapy that: improves bile flow, improves biliary secretion, reduces the production of hydrophobic primary bile acids cholic acid (CA) and chenodeoxycholic acid (CDCA), reduces the production of toxic bile acids (such as lithocholic acid (LCA) and deoxycholic acid (DCA)) and/or reduces the hydrophobicity of the bile acid pool.

Cholestasis refers to a condition in which the flow of bile from the liver is reduced or blocked, or in which there is a failure in bile flow. Bile flow failures may arise anywhere in the hepatic and biliary system. In general, cholestasis may be extrahepatic cholestasis, which occurs outside the liver cells, or may be intrahepatic cholestasis, which occurs inside the liver cells.

Extrahepatic cholestasis can result from benign biliary strictures, benign pancreatic disease cysts, diverticulitis, liver damage, common bile duct stones, pancreatitis, pancreatic cancer or pseudocyst, periampullary cancer, bile duct carcinoma, primary sclerosing cholangitis, or extrinsic duct compression, for example, compression due to a mass or tumor on a nearby organ.

Intrahepatic cholestasis can be caused by viral hepatitis including but not limited to Hepatitis B and C, sepsis, bacterial abscess, drugs e.g., drug-induced idiosyncratic hepatotoxicity, lymphoma, tuberculosis, metastatic carcinoma, sarcoidosis, amyloidosis, intravenous feeding, primary biliary cirrhosis, primary sclerosing cholangitis, alcoholic hepatitis with or without cirrhosis, chronic hepatitis with or without cirrhosis, pregnancy, Sjogren syndrome, nonalcoholic steatohepatitis, nonalcoholic fatty liver disease, chronic hepatitis with or without cirrhosis, intrahepatic cholestasis of pregnancy, PFIC, etc. Drug-induced cholestasis is the blockage of the flow of bile from the liver caused by medication, and may be caused by: gold salts, nitrofurantoin, anabolic steroids, oral contraceptives, chlorpromazine, prochlorperazine, sulindac, cimetidine, erythromycin, tobutamide, imipramine, ampicillin and other penicillin-based antibiotics, etc. Drug-induced cholestasis and hepatotoxicity are common obstacles to drug therapy in the clinic and pose major problems for drug development and for novel applications of approved drugs. Drug-induced cholestasis also accounts for 2-5% of patients hospitalized with jaundice, ˜10% of all cases of acute hepatitis, and over 50% of acute liver failure.

Cholestasis may also result from inherited cholestatic liver disease, from drug-induced cholestasis arising from certain drugs, and acute hepatotoxic reactions brought about by drugs and inflammatory conditions which impact liver function.

Portal hypertension refers to a disorder manifesting as increased pressure in the portal vein, which is the vein that conducts blood from the intestine to the liver. The increased pressure in the portal vein may be due to a variety of causes, including inflammation, fibrosis, splenic arteriovenous fistulae, splenic or portal vein thrombosis, massive splenomegaly, sarcoidosis, schistosomiasis, nodular regenerative hyperplasia, primary biliary cirrhosis, hepatitis, autoimmune disease, etc.

A biliary disorder according to the invention is any disorder arising, or potentially arising, from cholestasis, portal hypertension, or any disorder benefited by the administration of a combination therapy as described herein. Biliary disorders include without limitation benign biliary strictures, benign pancreatic disease cysts, diverticulitis, liver fibrosis, liver damage, common bile duct stones, pancreatitis, pancreatic cancer or pseudocyst, periampullary cancer, bile duct carcinoma, primary sclerosing cholangitis, autoimmune cholangitis, extrinsic duct compression (e.g., compression due to a mass or tumor on a nearby organ, viral hepatitis (e.g., Hepatitis A, B, C, D, E, herpes simplex, cytomegalovirus, Epstein-Barr, adenovirus), sepsis, bacterial abscess, use of drugs e.g., drug-induced idiosyncratic hepatotoxicity, lymphoma, tuberculosis, metastatic carcinoma, sarcoidosis, amyloidosis, intravenous feeding, primary biliary cirrhosis, primary sclerosing cholangitis, alcoholic hepatitis with or without cirrhosis, nonalcoholic steatohepatitis, nonalcoholic fatty liver disease, chronic hepatitis with or without cirrhosis, intrahepatic cholestasis of pregnancy, biliary calculosis, biliary dyscinesia, Sjogren syndrome, Wilson's disease, ischemia, acute liver failure, α1-antitrypsin deficiency, Progressive Familial Intrahepatic Cholestasis (PFIC) such as PFIC2, Benign Recurrent Intrahepatic Cholestasis (BRIC), hepatocellular carcinoma (HCC), portal hypertension, veno-occlusive disease, hepatic vein thrombosis, autoimmune hepatitis, etc.

Gastrointestinal Disorders

Gastrointestinal (GI) disorders include any disorder or condition that have as contributory factor inflammation of the gastrointestinal tract caused, or exacerbated, by bile acids. In some embodiments, a GI disorder is an inflammatory GI disorder, such as inflammation in the intestinal region of a subject. In some embodiments, an inflammatory GI disorder may include, without limitation, necrotizing enterocolitis (NEC), gastritis, ulcerative colitis, Crohn's disease, inflammatory bowel disease, irritable bowel syndrome, pseudomembranous colitis, gastroenteritis, radiation induced enteritis, chemotherapy induced enteritis, gastroesophageal reflux disease (GERD), peptic ulcer, non-ulcer dyspepsia (NUD), celiac disease, intestinal celiac disease, gastrointestinal complications following bariatric surgery, gastric carcinogenesis, or gastric carcinogenesis following gastric or bowel resection.

Bile Acids

Bile acids are amphipathic compounds derived from cholesterol and are a subclass of steroids. Bile acids and bile alcohols are steroids whose structure is related to cholane or cholestane; accordingly, bile acids and bile alcohols may be termed cholanoids (13). The term “bile acid” is a generic term for cholanoid molecules having a carboxyl group and does not denote an ionization state.

The term “bile salt” may be used for a salt in which the anion is a conjugated bile acid, an unconjugated bile acid, or a conjugate of a bile alcohol, or may be used as a generic term to include both conjugated bile acids and bile alcohol conjugates occurring in nature as water-soluble anions (13). For example, bile salts may be bile acids conjugated with glycine or taurine as sodium salts.

The bile acids may have various hydroxyl groups, such as dihydroxylated bile acids, trihydroxylated bile acids, tetrahydroxylated bile acids, and pentahydroxylated bile acids, e.g., cholic acid, ursodeoxycholic acid, nor-ursodeoxycholic acid, chenodeoxycholic acid, deoxycholic acid, muricholic acid.

The numbering system for the carbon atoms of the bile acid skeleton, as used herein, is as follows.

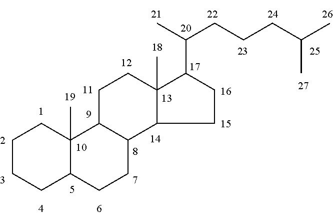 [SMILES: CC(C)CCCC(C)C1CCC2C3CCC4CCCCC4(C)C3CCC12C]

C24 bile acids are termed cholanoic acids or cholanoates, while C27 bile acids are termed cholestanic acids or cholestanoates. In general, the configuration of the side chain is 1713, with a 513 hydrogen (A/B ring junction in cis configuration). “Allo” bile acids are bile acids with a 5a hydrogen (13).

Bile acids may be polyhydroxylated. Polyhydoxylated bile acids, in accordance with the present disclosure, are those with four or more hydroxyl groups. Accordingly, a polyhydroxylated bile acid compound according to the invention includes without limitation tetrahydroxylated bile acids, pentahydroxylated bile acids, hexahydroxylated bile acids, etc., up to the maximum level of hydroxylation possible.

In some embodiments, a tetrahydroxylated bile acid may be a compound as represented in Formula I:

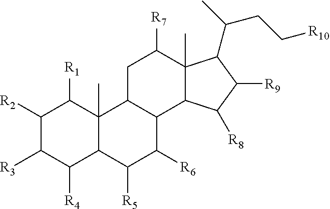

or a pharmaceutically-acceptable derivative thereof, in which any four of R₁ to R₉ may be —OH and Rio may be —COOH or —CH₂OH.

In some embodiments, any four of R₁ to R₉ may each independently be —OH, —F, —Cl, —Br, alkyl (for example, —CH₃, —CH₂—CH₃), —SO₄, or glucose and Rio may be —COOH or —CH₂OH.

In some embodiments, polyhydroxylated bile acids according to the invention are at least tetrahydroxylated i.e. have four or greater than four hydroxyl groups- In some embodiments, the hydroxyl groups are present on the steroid nucleus. In some embodiments, hydroxyl groups may also be present on the alkyl side chain.

A tetrahydroxylated bile acid according to the invention includes, without limitation, a 3,6,7,12-tetrahydroxycholanoic acid; a 3,4,7,12-tetrahydroxycholanoic acid; a 1,2,7,12-tetrahydroxycholanoic acid; a 1,3,7,12-tetrahydroxycholanoic acid; a 2,3,7,12-tetrahydroxycholanoic acid; a 3,7,16,24-tetrahydroxycholanoic acid; or a 3,7,15,24-tetrahydroxycholanoic acid, or derivatives thereof.

A 3,6,7,12-tetrahydroxycholanoic acid according to the invention includes, without limitation, a 3α, 6α, 7α, 12α-tetrahydroxy-5β-cholan-24-oic acid; a 3α, 6β, 7α, 12α-tetrahydroxy-5β-cholan-24-oic acid; a 3α, 6α, 7β, 12α-tetrahydroxy-5β-cholan-24-oic acid; a 3α, 6β, 7β, 12α-tetrahydroxy-5β-cholan-24-oic acid; a 3α, 6α, 7α, 12β-tetrahydroxy-5β-cholan-24-oic acid; a 3α, 6β, 7α, 12β-tetrahydroxy-5β-cholan-24-oic acid, or a 3α, 6β, 7β, 12β-tetrahydroxy-5β-cholan-24-oic acid, or derivatives thereof.

A 3,4,7,12-tetrahydroxycholanoic acid according to the invention includes, without limitation, a 3α, 4β, 7α, 12α tetrahydroxy-5β-cholan-24-oic acid, a 3α, 4α, 7α, 12α-tetrahydroxy-5β-cholanoic acid, or derivatives thereof.

A 1,3,7,12-tetrahydroxycholanoic acid according to the invention includes, without limitation, a 1β, 3α, 7α, 12α tetrahydroxy-5β-cholan-24-oic acid, or derivatives thereof.

A 2,3,7,12-tetrahydroxycholanoic acid according to the invention includes, without limitation, a 2β, 3α, 7α, 12α tetrahydroxy-5β-cholan-24-oic acid, a 2α, 3α, 7α, 12α-tetrahydroxy-5β-cholanoic acid, or derivatives thereof.

A 3,7,16,24-tetrahydroxycholanoic acid according to the invention includes, without limitation, a 3α, 7α, 16α, 24 tetrahydroxy-5β-cholane or derivatives thereof.

A 3,7,15,24-tetrahydroxycholanoic acid, according to the invention includes without limitation, a 3α, 7β, 15α, 24 tetrahydroxy-5β-cholane or derivatives thereof.

In alternative embodiments, polyhydroxylated bile acid compounds according to the invention include, without limitation, a 3α, 7α, 12α, 24 tetrahydroxy-5β-26-oic acid; a 3α, 7α, 12α, 24 tetrahydroxy-5β-cholest-25-ene; a 3α, 7α, 24, 26 tetrahydroxy-5β-Cholestane; or a 3α, 7α, 12α, 24, 26 pentahydroxy-5β-cholestane or derivatives thereof.

In alternative embodiments, polyhydroxylated bile acid compounds according to the invention are more hydrophilic than cholate (1, 16), as measured for example by the distribution and configurations of polar [OH⁻] and apolar (H⁺) residues along the steroid ring, or by retention times in reverse-phase HPLC (17). In some embodiments, polyhydroxylated bile acid compounds according to the invention have a hydrophobicity of less than 0.45, 0.40, 0.35, 0.30, 0.25, 0.20, 0.15, 0.10, or 0.05 relative to taurocholate (which is assigned a value of 1.0; see for example Asamoto et al. (18)).

The term “conjugated bile acid” may be used to indicate a bile acid conjugated to a group that gives additional hydrophilicity or charge to the molecule. In alternative embodiments, the polyhydroxylated (such as tetrahydroxylated) bile acid compounds according to the invention include taurine and/or glycine conjugates. In alternative embodiments, the polyhydroxylated (such as tetrahydroxylated) bile acid compounds according to the invention include conjugates with any other suitable amino acids. In alternative embodiments, the polyhydroxylated (such as tetrahydroxylated) bile acid compounds according to the invention include conjugates with sulfate, phosphate, Coenzyme A, glucuronate, glucose, xylose, and other sugars, N-acetylglucosamine, etc. For example, conjugated polyhydroxylated (such as tetrahydroxylated) compounds according to the invention include, without limitation, tauryl or glycyl conjugates of 3α, 6β, 7α, 12β-tetrahydroxy-5β-cholan-24-oic acids, tauryl or glycyl conjugates of 3α, 6β, 7β, 12β-tetrahydroxy-5β-cholan-24-oic acids, tauryl conjugates of 3α, 6β, 7α, 12α-tetrahydroxy-5β-cholan-24-oic acids, tauryl conjugates of 3α, 6β, 7β, 12α-tetrahydroxy-5β-cholan-24-oic acids, ethanesulfonic acid, 2-[(3,6,7,12-tetrahydroxy-24-oxocholan-24-yl)amino], e.g., ethanesulfonic acid, 2-[[(3α,5(3,6α,7α,12α)-3,6,7,12-tetrahydroxy-24-oxocholan-24-yl]amino]-, Glycine, N-(3,6,7,12-tetrahydroxy-24-oxocholan-24-yl) e.g., Glycine, N-[(3α,5β,6β,7(3,12α)-3,6,7,12-tetrahydroxy-24-oxocholan-24-yl], Glycine, N-[(3α,5β,6β,7α,12α)-3,6,7,12-tetrahydroxy-24-oxocholan-24-yl], Glycine, N-[(3α,5β,6α,7(3,12α)-3,6,7,12-tetrahydroxy-24-oxocholan-24-yl], Glycine, N-[(3α,5β,6α,7α,12α)-3,6,7,12-tetrahydroxy-24-oxocholan-24-yl], etc.

In alternative embodiments, the polyhydroxylated (such as tetrahydroxylated) bile acid compounds are conjugated with a group as described herein or known in the art at position 24 of the alkyl side chain. For example, a polyhydroxylated (such as tetrahydroxylated) bile acid compound may be conjugated with taurine or glycine at position 24 of the alkyl side chain of the polyhydroxylated (such as tetrahydroxylated) bile acid.

The polyhydroxylated (such as tetrahydroxylated) bile acid compounds according to the invention include isomers e.g., stereoisomers. For example, 3β and 5α hydroxy tetrahydroxycholanoic acid are included, as are any stereoisomeric configurations and combinations thereof.

The polyhydroxylated (such as tetrahydroxylated) bile acid compounds according to the invention include physiologically or pharmaceutically-acceptable derivatives, such as salts, esters, enol ethers, enol esters, solvates, hydrates and prodrugs of the compounds described herein. Pharmaceutically-acceptable salts, include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc, aluminum, and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

Compounds and salts thereof of this invention and for use in this invention are generally provided in substantially purified form. A compound or salt (if naturally occurring) is “substantially pure” or “isolated” when it is separated from the components that naturally accompany it (e. g, cells of a source organism or tissue). A compound may be substantially pure or isolated when it is substantially free of cellular contaminants, i.e, that it is present ex vivo and in a concentration greater than that of the compound in a source organism, tissue, or other natural source. Typically, a compound is substantially pure or isolated when it is at least 10%, 20%, 30%, 40%, 50%, or 60%, more generally 70%, 75%, 80%, or 85%, or over 90%, 95%, or 99% by weight, of the total material in a sample. Thus, for example, a compound that is chemically synthesized will generally be substantially free from its naturally associated components. A substantially pure compound can be obtained, for example, by extraction from a natural source or by chemical synthesis. A substantially pure compound may include stereoisomers or differentially hydroxylated mixtures. Purity can be measured using any appropriate method such as column, gas, or liquid chromatography or mass spectrometry.

In an alternative embodiment of the invention, a composition comprising a racemic mixture of a polyhydroxylated (such as tetrahydroxylated) bile acid is provided. The racemic mixture may be produced as a result of the chemical synthesis of the polyhydroxylated (such as tetrahydroxylated) bile acid; alternatively, two or more stereochemically pure enantiomers may be combined. In another embodiment, the composition may comprise two or more polyhydroxylated (such as tetrahydroxylated) bile acids.

Farnesoid-X Receptor Agonists

Farnesoid X receptor (FXR) is a nuclear receptor expressed at high levels in the liver, intestine, kidney, adrenal glands, and adipose tissue. FXR agonists include without limitation, obeticholic acid (OCA), the 6α-ethyl derivative of chenodeoxycholic acid (CDCA), cafestol, fexaramine, Cilofexor (2-[3-[2-chloro-4-[[5-cyclopropyl-3-(2,6-dichlorophenyl)-4-isoxazolyl]methoxy]phenyl]-3-hydroxy-1-azetidinyl]-4-pyridinecarboxylic acid), MET409 (Metacrine, Inc.), EDP-305 (Enanta Pharmaceuticals, Inc.), Tropifexor (2-[(1R,5S)-3-[[5-Cyclopropyl-3-[2-(trifluoromethoxy)phenyl]-1,2-oxazol-4-yl]methoxy]-8-azabicyclo[3.2.1]octan-8-yl]-4-fluoro-1,3-benzothiazole-6-carboxylic acid, LJN452, Novartis), LMB763 (Novartis), EYP001 (ENYO Pharma SA), EP-024297 (Enanta Pharmaceuticals, Inc.), AKN-083 (Allergan), RDX023 (Ardelyx, Inc.), AGN-242256 (Allergan), etc. Additional FXR agonists are described, for example, in WO/2012/087519, published Jun. 28, 2012, WO2017049173 published Mar. 23, 2017, and in WO2017049177 published Mar. 23, 2017.

Preparation of Polyhydroxylated Bile Acids

THBA compounds according to the invention, or for use according to the invention, including pharmaceutically acceptable salts or derivatives thereof, may be obtained by synthesis making use of common procedures as exemplified herein or known in the art. Such synthetic THBA compounds can, optionally, be labeled or derivatized for analytical or drug development purposes. The THBA compounds may be synthesized using standard techniques such as those described in Tohma et al., 1985 (19); Iida et al, 1991a (20); Iida et al., 1991b (21); Aggarwal et al., 1992 (22); Iida et al., 1993 (23); Kurosawa et al, 1995 (24); Kurosawa et al., 1996 (25); Iida et al, 2002 (26); Tserng K Y and Klein P D (1977) (27), Leppik R A (1983) (28), or Iida T. et al. (1990) (29, 30) etc., all of which are specifically incorporated by reference. For example, THBAs may be prepared as indicated in Tohma et al., 1985 (19); Iida et al., 1991b (21); Aggarwal et al., 1992 (22); Iida et al., 1993 (23); Kurosawa et al., 1996 (25); or Iida et al, 2002 (26). The THBA compounds may be synthesized as, for example, described in WO 2011/022838, published Mar. 3, 2011. Pentahydroxy bile acids may be prepared, for example, as indicated in Kurosawa et al., 1996 (25).

Preparation of Farnesoid-X Receptor Agonists

FXR agonists according to the invention, or for use according to the invention, including pharmaceutically acceptable salts or derivatives thereof, may be obtained by synthesis making use of common procedures as exemplified herein or known in the art. Such synthetic FXR agonists compounds can, optionally, be labeled or derivatized for analytical or drug development purposes. FXR agonists may be obtained from commercial sources or prepared as described, for example, in 31; 32)

Pharmaceutical Compositions, Dosages, and Administration

Polyhydroxylated bile acids, such as tetrahydroxylated bile acids, can be provided in combination with FXR agonists (the “claimed combination” or the “combination therapy” in accordance with the present disclosure).

In some embodiments, the amount of the FXR agonist can be an inoperable dose, for example a subtherapeutic dose, a subclinical dose, or a toxic dose. It is to be understood that an inoperable dose may vary depending on the subject or patient (e.g., adult, pediatric, geriatric or a subject or patient have a comorbidity that can affect dosage regimens).

In some embodiments, the FXR agonist is provided at a lower dose than the bile acid.

In some embodiments, the ratio of the FXR agonist, such as OCA or tropifexor, to the polyhydroxylated bile acid, such as a tetrahydroxylated bile acid, in the claimed combination can be equal to or less than about 1:100 (w/w). In some embodiments, the ratio of the FXR agonist, such as OCA or tropifexor, to the polyhydroxylated bile acid, such as a tetrahydroxylated bile acid, in the claimed combination can be equal to or less than about 1:300 (w/w). In some embodiments, the ratio of the FXR agonist, such as OCA or tropifexor, to the polyhydroxylated bile acid, such as a tetrahydroxylated bile acid, in the claimed combination can be equal to or less than about 1:500 (w/w). In some embodiments, the ratio of the FXR agonist, such as OCA or tropifexor, to the polyhydroxylated bile acid, such as a tetrahydroxylated bile acid, in the claimed combination can be equal to or less than about 1:1000 (w/w). In some embodiments, the ratio of the FXR agonist, such as OCA or tropifexor, to the polyhydroxylated bile acid, such as a tetrahydroxylated bile acid, in the claimed combination can be equal to or less than about 1:100000 (w/w). In some embodiments, the ratio of the FXR agonist, such as OCA or tropifexor, to the polyhydroxylated bile acid, such as a tetrahydroxylated bile acid, in the claimed combination can be about 1:100 to about 1:300 (w/w), or any value in between. In some embodiments, the ratio of the FXR agonist, such as OCA or tropifexor, to the polyhydroxylated bile acid, such as a tetrahydroxylated bile acid, in the claimed combination can be about 1:50 to about 1:500 (w/w), or any value in between. In some embodiments, the ratio of the FXR agonist, such as OCA or tropifexor, to the polyhydroxylated bile acid, such as a tetrahydroxylated bile acid, in the claimed combination can be greater than 1:30. In some embodiments, the ratio of the FXR agonist, such as OCA or tropifexor, to the polyhydroxylated bile acid, such as a tetrahydroxylated bile acid, in the claimed combination can be greater than 1:30 to equal to or less than about 1:100000 (w/w), or any value in between.

In some embodiments, the ratio of the FXR agonist, such as OCA or tropifexor, to the polyhydroxylated bile acid, such as a tetrahydroxylated bile acid, in the claimed combination can be equal to or less than 1:100 (w/w). In some embodiments, the ratio of the FXR agonist, such as OCA or tropifexor, to the polyhydroxylated bile acid, such as a tetrahydroxylated bile acid, in the claimed combination can be equal to or less than 1:300 (w/w). In some embodiments, the ratio of the FXR agonist, such as OCA or tropifexor, to the polyhydroxylated bile acid, such as a tetrahydroxylated bile acid, in the claimed combination can be equal to or less than 1:500 (w/w). In some embodiments, the ratio of the FXR agonist, such as OCA or tropifexor, to the polyhydroxylated bile acid, such as a tetrahydroxylated bile acid, in the claimed combination can be equal to or less than 1:1000 (w/w). In some embodiments, the ratio of the FXR agonist, such as OCA or tropifexor, to the polyhydroxylated bile acid, such as a tetrahydroxylated bile acid, in the claimed combination can be equal to or less than 1:100000 (w/w). In some embodiments, the ratio of the FXR agonist, such as OCA or tropifexor, to the polyhydroxylated bile acid, such as a tetrahydroxylated bile acid, in the claimed combination can be 1:100 to 1:300 (w/w), or any value in between. In some embodiments, the ratio of the FXR agonist, such as OCA or tropifexor, to the polyhydroxylated bile acid, such as a tetrahydroxylated bile acid, in the claimed combination can be 1:50 to 1:500 (w/w), or any value in between. In some embodiments, the ratio of the FXR agonist, such as OCA or tropifexor, to the polyhydroxylated bile acid, such as a tetrahydroxylated bile acid, in the claimed combination can be greater than 1:30. In some embodiments, the ratio of the FXR agonist, such as OCA or tropifexor, to the polyhydroxylated bile acid, such as a tetrahydroxylated bile acid, in the claimed combination can be greater than 1:30 to equal to or less than 1:100000 (w/w), or any value in between.

In some embodiments, the ratio of the FXR agonist to the bile acid in the claimed combination can be less than the levels required for co-crystallization.

In some embodiments, the polyhydroxylated bile acid, such as a tetrahydroxylated bile acid, can be administered in a range from about 50 mg per adult subject per day to about 5000 mg per adult subject per day.

In some embodiments, the FXR agonist, for example OCA or tropifexor, can be administered at less than about 1 mg per adult subject per day.

In some embodiments, the FXR agonist can be administered at a dose at or below the minimal therapeutically effective dose or dose which exhibits no therapeutic benefit in the treatment of a GI or biliary disorder (a “subtherapeutic” dose). In some embodiments, the FXR agonist can be administered at a dose below the minimal dose used in standard clinical practice in the treatment of a GI or biliary disorder (a “subclinical” dose). For example, OCA may be administered at a dose less than about 5-about 10 mg per day or any value in between (about 74 ug/kg daily for a person of about 70 kg body weight); EDP-305 may be administered at a dose less than about 1 mg per day (about 14 ug/kg daily for a person of about 70 kg body weight); Cilofexor may be administered at a dose less than about 30 mg per day (about 0.45 mg/kg daily for a person of about 70 kg body weight); tropifexor may be administered at a dose less than about 0.01 mg per day (about 0.15 ug/kg daily for a person of about 70 kg body weight); or LMB763 may be administered at a dose less than about 5 mg per day (about 74 ug/kg daily for a person of about 70 kg body weight).

In some embodiments, the FXR agonist can be administered at less than or equal to one-fifth of the standard recommended dosage for that compound. For example, OCA may be administered at less than or equal to one-fifth of about 5-about 10 mg per day or any value in between (about 74-about 148 ug/kg daily for a person of about 70 kg body weight); EDP-305 may be administered at less than or equal to one-fifth of about 1-about 2.5 mg per day or any value in between (about 14-about 36 ug/kg daily for a person of about 70 kg body weight); Cilofexor may be administered at less than or equal to one-fifth of about 30-about 100 mg per day or any value in between (about 0.45-about 1.5 mg/kg or any value in between daily for a person of about 70 kg body weight); tropifexor may be administered at less than or equal to one-fifth of about 0.01-about 0.03 mg per day or any value in between (about 0.15-about 0.45 ug/kg daily for a person of about 70 kg body weight); or LMB763 may be administered at less than or equal to one-fifth of about 5 mg per day (about 74 ug/kg daily for a person of about 70 kg body weight).

In some embodiments, the claimed composition may include 3α, 6α, 7α, 12α-tetrahydroxy-5β-cholan-24-oic acid at a dosage of about 15 mg/Kg/day in combination with OCA at a dosage of about 0.05 mg/Kg/day (33; 34).

In some embodiments, the claimed composition may include 3α, 6α, 7α, 12α-tetrahydroxy-5β-cholan-24-oic acid at a dosage of about 15 mg/Kg/day in combination with tropifexor at a dosage of about 0.0004 mg/Kg/day.

In some embodiments the amount of FXR agonist, such as OCA or tropifexor, in the claimed composition may be substantially less than the amount used in current therapeutic practice. For example, adult human patients are dosed with OCA at about 5 mg per week to about 10 mg per day, or any value in between, depending on how well the dose is tolerated, whether the patient has decompensated liver disease and/or whether the patient is also taking UDCA. In another example, adult human patients are dosed with tropifexor at about 3 mg per week to about 0.03 mg per day, or any value in between, depending on how well the dose is tolerated.

By “about” is meant a variance (plus or minus) from a value or range of 5% or less, for example, 0.5%, 1%, 1.5%, 2.0%, 2.5%, 3.0%, 3.5%, 4.0%, 4.5%, 5.0%, etc.

In some embodiments, the FXR agonist, such as OCA or tropifexor, may be used at a dosage “less than an effective dose.” By “less than an effective dose” is meant a dose that is less than the dose used in current therapies or which exhibits no therapeutic benefit e.g., a subtherapeutic dose, a subclinical dose. In some embodiments, “less than an effective dose” is meant a dose that is less effective or ineffective when used as a monotherapy, or in combination with UDCA, or in combination with a compound other than a bile acid, when treating a biliary or GI disorder. In some embodiments, a dose that is less effective is a dose that is 5% to 99% percent, or any value in between, such as 5%, 10%, 20%, 30%, 40%, 50%, 60%, 70%, 80%, or 90%, less effective when used as a monotherapy, or in combination with UDCA, or in combination with a compound other than a bile acid, when treating a biliary or GI disorder, compared to the same dose when combined with a polyhydroxylated bile acid, such as a THBA.

In some embodiments, the FXR agonist, such as OCA or tropifexor, may be used at a “toxic dose.” By “toxic dose” is meant a dose or amount that results in an unacceptable toxicity level, when used as a monotherapy, or in combination with UDCA, or in combination with a compound other than a bile acid, when treating a biliary or GI disorder, compared to the same dose or amount when combined with a polyhydroxylated bile acid, such as a THBA, as described herein. In some embodiments, a toxic dose or amount is that which increases the level of one or more liver indicators, such as bilirubins, ALP (alkaline phosphatase), ALT (alanine aminotransferase), AST (aspartate aminotransferase), γ-GT (Gamma-Glutamyl Transpeptidase), etc. to outside the normal clinical range in a subject or which further increases the level of one or more liver indicators, such as bilirubins, ALP (alkaline phosphatase), ALT (alanine aminotransferase), AST (aspartate aminotransferase), γ-GT (Gamma-Glutamyl Transpeptidase), etc. in a subject having higher levels of the one or more liver indicators. In some embodiments, the level of increase of liver indicators, such as bilirubins, ALP (alkaline phosphatase), ALT (alanine aminotransferase), AST (aspartate aminotransferase), γ-GT (Gamma-Glutamyl Transpeptidase), etc. can be about 5% to about 100% percent, or any value in between, such as 5%, 10%, 20%, 30%, 40%, 50%, 60%, 70%, 80%, 90%, or 100% increase. In some embodiments, the level of increase of liver indicators, such as bilirubins, ALP (alkaline phosphatase), ALT (alanine aminotransferase), AST (aspartate aminotransferase), γ-GT (Gamma-Glutamyl Transpeptidase), etc. can be over 100% percent, compared to normal clinical values or ranges. Normal clinical values and ranges for liver indicators are well known. In some embodiments, the normal clinical ranges for selected liver indicators may be as follows: Bilirubin: 2 to 17 micromoles/L; ALP (alkaline phosphatase): 30 to 120 IU/L; ALT (alanine aminotransferase): 0 to 45 IU/L; AST (aspartate aminotransferase): 0 to 35 IU/L; γ-GT (Gamma-Glutamyl Transpeptidase): 0 to 30 IU/L.

An “effective amount” of a combination according to the invention includes a therapeutically effective amount or a prophylactically effective amount or a nutritionally effective amount. A “therapeutically effective amount” refers to an amount effective, at dosages and for periods of time necessary, to achieve the desired therapeutic result, such as increased bile flow, relief of jaundice, or improved liver functions as indicated by normalization of serum liver biochemical indicators, such as the levels of bilirubins, ALP (alkaline phosphatase), ALT (alanine aminotransferase), AST (aspartate aminotransferase), γ-GT (Gamma-Glutamyl Transpeptidase), etc. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as increased bile flow or improved liver functions as indicated by liver biochemical indicators, increased bile flow, relief of jaundice, or improved liver functions as indicated by normalization of serum liver biochemical indicators, such as the levels of bilirubins, ALP (alkaline phosphatase), ALT (alanine aminotransferase), AST (aspartate aminotransferase), γ-GT (Gamma-Glutamyl Transpeptidase), etc. Typically, a prophylactic dose is used in subjects prior to or at an earlier stage of disease, so that a prophylactically effective amount may be less than a therapeutically effective amount. An exemplary range for therapeutically or prophylactically effective amounts of a compound may be about 5-about 50 mg/day/kg of body weight of the subject e.g., a human. A “nutritionally effective amount” refers to an amount effective, at dosages and for periods of time necessary, to achieve the desired result, such as increased bile flow or improved liver functions as indicated by liver biochemical indicators.

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. In some embodiments, the polyhydroxylated bile acid, such as tetrahydroxylated bile acid, can be provided in combination with FXR agonist in a single formulation. In an alternative embodiment, the polyhydroxylated bile acid, such as tetrahydroxylated bile acid, can be provided in a separate formulation from the FXR agonist either simultaneously or within a suitable period time, for example, within 24 hours. It may be advantageous to formulate parenteral compositions in dosage unit form for ease of administration and uniformity of dosage.

In general, the combination therapy should be used without causing substantial toxicity. Toxicity of the combination therapy can be determined using standard techniques, for example, by testing in cell cultures or experimental animals or subjects and determining the therapeutic index, i.e., the ratio between the LD50 (the dose lethal to 50% of the population) and the ED50 (the minimum effective dose for 50% of the population) for non-human animals or the ratio between the TD50 (the dose toxic to 50% of the population) and the ED50 (the minimum effective dose for 50% of the population) for humans. Other methods that may be used to determine toxicity of the compounds of the invention include, but are not limited to, histological abnormality by H&E staining, trichrome staining or the like; changes in bile flow rate, and/or clearance of other bile substances (for example, as determined by bile duct cannulation); HPLC analysis, enzymatic assays or the like; changes in liver indicator profiles, for example level of bilirubins, level of ALP (alkaline phosphatase), level of ALT (alanine aminotransferase), level of AST (aspartate aminotransferase), level of γ-GT (Gamma-Glutamyl Transpeptidase), or the like. The maximum tolerated dose (MTD) is the highest regularly administered dose of a compound or composition that does not cause overt toxicity (e.g. does not cause unacceptable side effects) in a subject study over a period of time. The subject may be a human, or an animal, such as a mouse or a rat, for example. The regularly administered dose may be a daily dose, administered as a single bolus; alternately the daily dose may be divided into two or more partial doses so that the subject receives the total daily dose over time. The period of time of the study may vary from a few days to a few months, for example about 10, 20, 30, 60, 90 or 120 days, or any value therebetween. Examples of overt toxicity may include, but are not limited to, appreciable death of cells or organ dysfunction, toxic manifestations that are predicted materially to reduce the life span of the subject, or 10% or greater retardation of body weight gain. In some embodiments, the claimed combination may be provided together with other compounds (for example, nucleic acid molecules, small molecules, peptides, or peptide analogues), in the presence of a liposome, an adjuvant, or any pharmaceutically or physiologically acceptable carrier, in a form suitable for administration to humans or animals. If desired, treatment with the claimed combination according to the invention may be combined with more traditional and existing therapies for biliary disorders or disorders resulting in or potentially resulting in hepatotoxicity, or with existing nutritional supplements for stimulating bile flow, or for GI disorders. In some embodiments, the claimed combination according to the invention is administered where the approved therapeutic agent for cholestasis, ursodeoxycholate, is ineffective. In some embodiments, the claimed combination according to the invention is administered together with ursodeoxycholate or a variant or derivative thereof (e.g., sulfated ursodeoxycholate, glycoursodeoxycholate, tauroursodeoxycholate, etc.), Rifampicin, or any compound useful for treating cholestasis or portal hypertension or a GI disorder, or for stimulating bile flow.

Conventional pharmaceutical or nutritional supplement formulation practice may be employed to provide suitable formulations or compositions to administer the claimed combination to patients suffering from or presymptomatic for biliary disorders or disorders resulting in or potentially resulting in hepatotoxicity, or with existing nutritional supplements for stimulating bile flow, or for GI disorders. Any appropriate route of administration may be employed, for example, parenteral, intravenous, subcutaneous, intramuscular, intracranial, intraorbital, ophthalmic, intraventricular, intracapsular, intraspinal, intracisternal, intraperitoneal, intranasal, aerosol, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found in, for example, Remington's Pharmaceutical Sciences (35). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

For therapeutic or prophylactic compositions, the claimed combination may be administered to a subject in an amount sufficient to stop or slow cholestasis or to maintain or increase bile flow or to ameliorate portal hypertension. For nutritional supplements, the claimed combination may be administered to a subject in an amount sufficient to stimulate bile flow.

In some embodiments, the claimed combination can also provide therapeutic benefit to patients suffering from inherited cholestatic liver disease, from drug-induced cholestasis arising from the Bile Salt Export Pump (BSEP)-inhibitory activity of certain drugs, or from other biliary disorders, and can help alleviate acute hepatotoxic reactions brought about by drugs and inflammatory conditions which impact biliary function. In some embodiments, the claimed combination can be used in combination with a compound that does not inhibit BSEP. In some embodiments, the claimed combination can be used in combination with a compound that does not exhibit a preferential affinity for BSEP over P-glycoprotein (Mdr1). In some embodiments, the claimed combination can be used under conditions where BSEP is functional, for example, in combination with ursodeoxycholate.

In some embodiments, the claimed composition can enhance the therapeutic effect of an FXR agonist. Accordingly, in some embodiments, the present disclosure provides a method for enhancing the therapeutic effect of a farnesoid X receptor agonist by administering a polyhydroxylated bile acid in combination with an inoperable amount of a farnesoid X receptor agonist to a subject in need thereof. The enhancement can be compared, for example, in comparison to the efficacy of the farnesoid X receptor agonist when administered as a monotherapy.

Articles of Manufacture

Articles of manufacture containing packaging material and the claimed combination are provided.

Kits

A kit including the claimed composition, along with instructions for use of the compound or composition, is provided. The kit may be useful for treating a biliary disorder or GI disorder in a subject, and the instructions may include, for example, dose concentrations, dose intervals, preferred administration methods or the like.

The present invention will be further illustrated in the following examples.

### Example 1

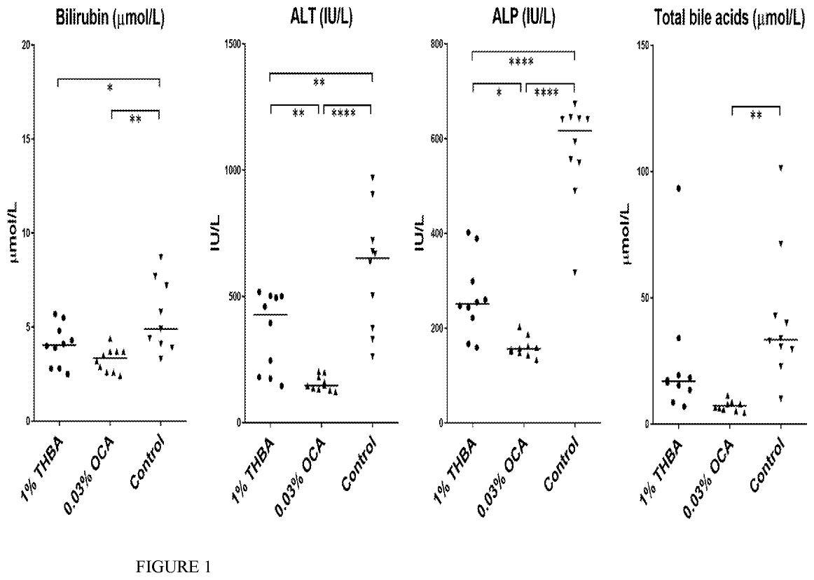

In a first study, a 1% THBA (3α, 6α, 7α, 12α-tetrahydroxy-5(3-cholan-24-oic acid) and 0.03% OCA were fed to a mouse model of human PSC and PFIC (female mice, starting at 3 weeks of age) for 17 weeks in a form of dietary supplementation (w/w), either individually or in combination. The body weight and food consumption were recorded weekly during treatment. After the treatment, mice were euthanized and tissues/organs were sampled for further analysis. In either group of 0.03% OCA, or 1% THBA, mono-treatment from 3 to 20 weeks of feeding, we observed significantly improved liver indicator profiles with no significant, obvious, adverse effects (FIG. 1). However, when applied in combination, the Mdr2^{-/-} mice of treatment age, fed OCA at 0.03% and THBA at 1%, started losing weight and became jaundiced as early as 4 days after the start of treatment, and started dying unexpectedly. This combination treatment was terminated due to these unexpected adverse effects.

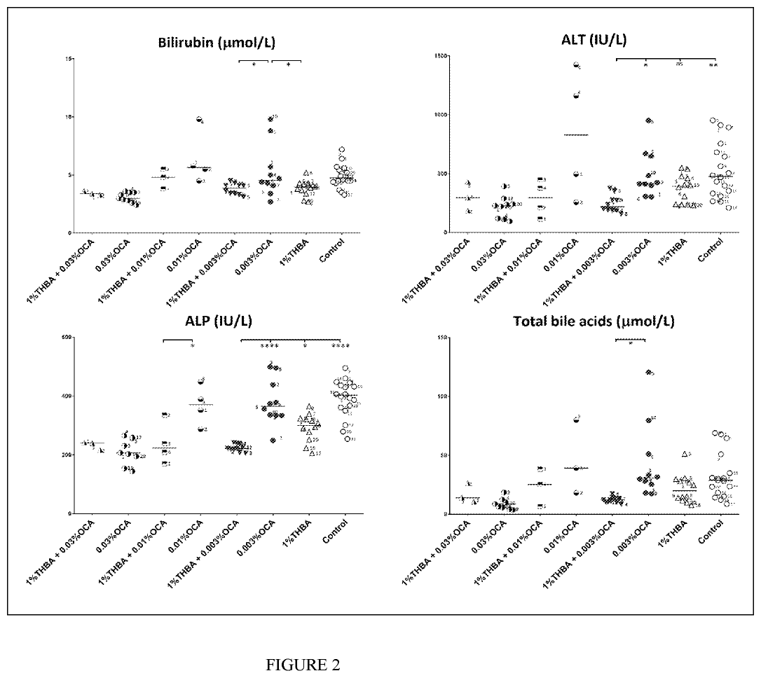

In a follow-up study, we tested different doses of OCA at 0.03%, 0.01% and 0.003%, alone or in combination with 1% THBA at 8 weeks of age, for 4 weeks. The treatment group fed OCA at 0.03% in combination with THBA at 1% (w/w) exhibited an adverse response, in which two out of five mice had to be terminated before the endpoint. Female Mdr2^{-/-} mice, fed a 10 times lower dose of OCA (0.003% w/w in diet), by itself did not show any treatment effect. However, 0.003% OCA in combination with 1% THBA, produced a treatment efficacy superior to that of either mono-treatment (FIG. 2). The combination treatment, in this study, resulted in reduced alkaline phosphatase (ALP) levels in these mice, suggesting reduced cholangiocyte injury.

### Example 2

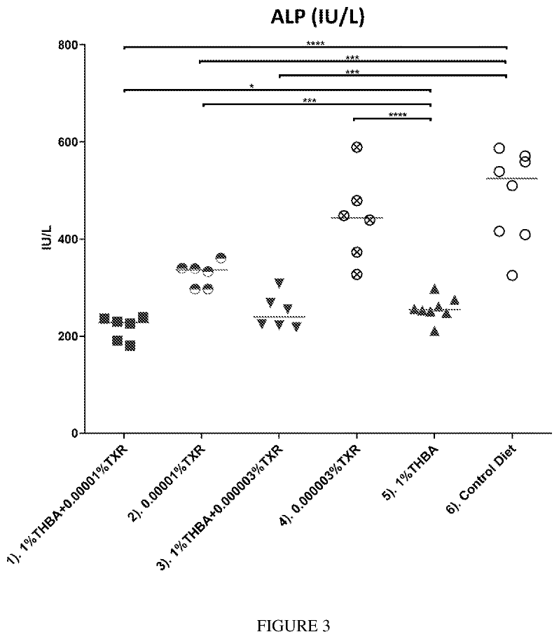

In another study, a 1% THBA (3α, 6α, 7α, 12α-tetrahydroxy-5(3-cholan-24-oic acid) and tropifexor (TXR) were fed to female Mdr2^{-/-} mice, of around 12 weeks of age, for 26-28 days in a form of dietary supplementation (w/w), either individually or in combination. The body weight and food consumption were recorded weekly during treatment. After the treatment, mice were euthanized and tissues/organs were sampled for further analysis. Dietary supplementations were 1). 1% THBA+0.00001% TXR; 2). 0.00001% TXR only; 3) 1% THBA+0.000003% TXR; 4) 0.000003% TXR; 5) 1% THB A only; and 6) the control diet. Treatment with THBA and TXR reduced liver injury, caused by toxic bile acid in the bile due to a lack of phospholipids in the bile of Mdr2^{-/-} mice, as demonstrated by reduced ALP levels in the plasma of the female Mdr2^{-/-} mice (FIG. 3).

### Other Embodiments

The present invention has been described with regard to one or more embodiments. However, it will be apparent to persons skilled in the art that a number of variations and modifications can be made without departing from the scope of the invention as defined in the claims. Therefore, although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the specification, the word “comprising” is used as an open-ended term, substantially equivalent to the phrase “including, but not limited to,” and the word “comprises” has a corresponding meaning. It is to be however understood that, where the words “comprising” or “comprises,” or a variation having the same root, are used herein, variation or modification to “consisting” or “consists,” which excludes any element, step, or ingredient not specified, or to “consisting essentially of” or “consists essentially of,” which limits to the specified materials or recited steps together with those that do not materially affect the basic and novel characteristics of the claimed invention, is also contemplated. Citation of references herein shall not be construed as an admission that such references are prior art to the present invention. All publications are incorporated herein by reference as if each individual publication was specifically and individually indicated to be incorporated by reference herein and as though fully set forth herein. The invention includes all embodiments and variations substantially as hereinbefore described and with reference to the examples and drawings.

### REFERENCES

1. Hofmann, A. F. (1999) Arch Intern Med 159, 2647-58.
2. Rust C, Karnitz L M, Paya C V, Moscat J, Simari R D, Gores G J. J Biol Chem. 2000; 275:20210-20216
3. Sodeman T, Bronk S F, Roberts P J, Miyoshi H, Gores G J. Am J Physiol Gastrointest Liver Physiol. 2000; 278:G992-G999
4. Jones B A, Rao Y P, Stravitz R T, Gores G J. Am J Physiol. 1997; 272:G1109-G1115
5. Kwo P, Patel T, Bronk S F, Gores G J. Am J Physiol. 1995; 268:G6β-G621
6. Rodrigues C M, Fan G, Ma X, Kren B T, Steer C J. J Clin Invest. 1998; 101:2790-2799
7. Wang H, Chen J, Hollister K, Sowers L C, Forman B M. Mol Cell. 1999; 3:543-553
8. Makishima M, Okamoto A Y, Repa J J, Tu H, Learned R M, Luk A, Hull M V, Lustig K D, Mangelsdorf D J, Shan B. Science. 1999; 284:1362-1365
9. Chiang J Y, Kimmel R, Weinberger C, Stroup D. J Biol Chem. 2000; 275:10918-10924
10. Parks D J, Blanchard S G, Bledsoe R K, Chandra G, Consler T G, Kliewer S A, Stimmel J B, Willson T M, Zavacki A M, Moore D D, et al. Science. 1999; 284:1365-1368
11. Goodwin B, Jones S A, Price R R, Watson M A, McKee D D, Moore L B, Galardi C, Wilson J G, Lewis M C, Roth M E, et al. Mol Cell. 2000; 6:517-526
12. Sinal C J, Tohkin M, Miyata M, Ward J M, Lambert G, Gonzalez F J. Cell. 2000; 102:731-744
13. Hofmann A F, Sjovall J, Kurz G, Radominska A, Schteingart C D, Tint G S, Vlahcevic Z R, Setchell K D. J Lipid Res. 1992; 33(4):599-604.
14. Jansen P L and Sturm E. Liver Int 2003; 23(5):315-22
15. Feng, C. Clarification of Obeticholic Acid Dosing: Response to “Occurrence of Jaundice Following Simultaneous Ursodeoxycholic Acid Cessation and Obeticholic Acid Initiation” by Quigley et al. Dig Dis Sci. 2018; 63(7): 1980-1981. Published online 2018 May 10).
16. Hofmann, A. F. (2001) Hepatology 34, 848-50.
17. Bohme, M., Muller, M., Leier, I., Jedlitschky, G. and Keppler, D. (1994) Cholestasis caused by inhibition of the adenosine triphosphate-dependent bile salt transport in rat liver. Gastroenterology 107, 255-265
18. Asamoto, Y., Tazuma, S., Ochi, H., Chayama, K. & Suzuki, H. (2001) Biochem J 359, 605-10.
Tohma, M, Mahara R, Takeshita H, Kurosawa T, Ikegawa S, Nittono H. Chem. Pharm. Bull. 1985; 33(7):3071-3073
20. Iida T, Tamaru T, Chang F C, Goto J, Nambara T. Journal of Lipid Research 1991; 32:649-658
21. Iida T, Komatsubara I, Chang F C, Goto J, Nambara T. Steroids 1991; 56:114-122
22. Aggarwal S K, Batta A K, Salen G, Shefer S. Steroids 1992; 57:107-111
23. Iida T, Nishida S, Chang F C, Niwa T, Goto J, Nambara T. Steroids 1993; 58:148-152
24. Kurosawa T, Nakano H, Sato M, Tohma M. Steroids 1995; 60:439-444
25. Kurosawa T, Sato M, Nakano H, Tohma M. Steroids 1996; 61:421-428
26. lida T, Hikosaka M, Kakiyama G, Shiraishi K, Schteingart C D, Hagey L R, Ton-Nu H T, Hofmann A F, Mano N, Goto J, Nambara T. Chem. Pharm. Bull. 2002; 50(10):1327-1334
27. Tserng K Y and Klein P D. “Formylated bile acids: improved synthesis, properties, and partial deformylation.” 1977 Steroids, 29: 635-648
28. Leppik RA. “Improved synthesis of 3-keto, 4-ene-3-keto, and 4,6-diene-3-keto bile acids.” 1983 Steroids, 41: 475-484
29. Iida T. et al. “Potential bile acid metabolites. 17. Synthesis of 2 beta-hydroxylated bile acids.” 1991 Steroids, 56: 114-122
30. Iida T. et al. “Potential bile acid metabolites. 116. Synthesis of stereoisomeric 3 alpha,6,7,12 alpha-tetrahydroxy-5 beta-cholanoic acids.”1990 Steroids, 55: 530-539.
31. (1. PMID: 27468093. Obeticholic acid for the treatment of primary biliary cholangitis. Ali A H, Lindor K D. Expert Opin Pharmacother. 2016 September; 17(13):1809-15. doi: 10.1080/14656566.2016.1218471. Epub 2016 Aug. 9. Review.
32. 2. PMID: 29649907: Farnesoid X receptor modulators 2014-present: a patent review. Sepe V, Distrutti E, Fiorucci S, Zampella A. Expert Opin Ther Pat. 2018 May; 28(5):351-364. doi: 10.1080/13543776.2018.1459569. Epub 2018 Apr. 13.)
33. Nair A B, Jacob S. A simple practice guide for dose conversion between animals and human. J Basic Clin Pharm. 2016 March; 7(2):27-31. doi: 10.4103/0976-0105.177703. Review. PubMed PMID: 27057123
34. Verma A, Jazrawi R P, Ahmed H A, Davis T, Bland J M, Benson M, Orchard R T, Theodossi A, Maxwell J D, Northfield T C Optimum dose of ursodeoxycholic acid in primary biliary cirrhosis. Eur J Gastroenterol Hepatol, 1999 October; 11(10):1069-76 PubMed Central PMCID: PMC4804402
35. “Remington's Pharmaceutical Sciences” (19th edition), ed. A. Gennaro, 1995 (Ref), Mack Publishing Company, Easton, Pa.
36. Claudel et al. The Farnesoid X Receptor: A Molecular Link Between Bile Acid and Lipid and Glucose Metabolism Arterioscler Thromb Vasc Biol 2005 October; 25(10):2020-30.

1. A pharmaceutical composition comprising:
i) a polyhydroxylated bile acid; and
ii) an inoperable amount of a farnesoid X receptor agonist,
in combination with a pharmaceutically acceptable carrier.

2. The pharmaceutical composition of claim 1 wherein the inoperable amount of a farnesoid X receptor agonist is a subtherapeutic amount, a subclinical amount or a toxic amount.

3. The pharmaceutical composition of claim 1 wherein the ratio of the farnesoid X receptor agonist to the polyhydroxylated bile acid is equal to or less than 1:100.

4. The pharmaceutical composition of claim 1 wherein the farnesoid X receptor agonist is obeticholic acid or tropifexor.

5. The pharmaceutical composition of claim 1 wherein the polyhydroxylated bile acid is a tetrahydroxylated bile acid.

6. The pharmaceutical composition of claim 5 wherein the tetrahydroxylated bile acid is 3α, 6α, 7α, 12α-tetrahydroxy-5β-cholan-24-oic acid.

7. The pharmaceutical composition of claim 5 wherein the tetrahydroxylated bile acid is a conjugated compound.

8. The pharmaceutical composition of claim 7 wherein the conjugated compound is a taurine or a glycine conjugate.

9. A method of treating a biliary disorder or a gastrointestinal disorder comprising administering the pharmaceutical composition of claim 1 to a subject in need thereof.

10. The method of claim 9 wherein the biliary disorder arises from cholestasis.

11. The method of claim 9 wherein the gastrointestinal disorder is an inflammatory disorder.

12. A method for reducing the toxicity, or enhancing the therapeutic effect, of a farnesoid X receptor agonist, the method comprising administering:
i) a polyhydroxylated bile acid; and
ii) a toxic amount, or an inoperable amount, of the farnesoid X receptor agonist,
in combination with a pharmaceutically acceptable carrier, to a subject in need thereof.

13. (canceled)

14. The method of claim 12 wherein the inoperable amount of a farnesoid X receptor agonist is a subtherapeutic amount, a subclinical amount or a toxic amount.

15. The method of claim 12 wherein the ratio of the farnesoid X receptor agonist to the polyhydroxylated bile acid is equal to or less than 1:100.

16. The method of claim 12 wherein the farnesoid X receptor agonist is obeticholic acid or tropifexor.

17. The method of claim 12 wherein the polyhydroxylated bile acid is a tetrahydroxylated bile acid.

18. The method of claim 17 wherein the tetrahydroxylated bile acid is 3α, 6α, 7α, 12α-tetrahydroxy-5β-cholan-24-oic acid.

19. The method of claim 17 or 18 wherein the tetrahydroxylated bile acid is a conjugated compound, wherein the conjugated compound is a taurine or a glycine conjugate.

20. (canceled)

21. The method of claim 12 wherein the subject is a human.

22. (canceled)

23. (canceled)

24. An article of manufacture comprising the pharmaceutical composition of claim 1, together with instructions for treating a biliary disorder or a gastrointestinal disorder.
